(19) United States
(12) Patent Application Publication
(10) Pub. No.: US 2026/0210968 A1
(43) Pub. Date: Jul. 23, 2026

(54) IDENTIFICATION AND MONITORING OF MONOCLONAL IMMUNOGLOBULINS BY MOLECULAR MASS

(71) Applicant: MAYO FOUNDATION FOR MEDICAL EDUCATION AND RESEARCH, Rochester, MN (US)

(72) Inventors: David R. Barnidge, Rochester, MN (US); David L. Murray, Rochester, MN (US)

(21) Appl. No.: 19/447,644

(22) Filed: Jan. 13, 2026

Related U.S. Application Data
(63) Continuation of application No. 14/777,236, filed on Mar. 10, 2014, now Pat. No. 12,546,782.
(60) Provisional application No. 61/792,944, filed on Mar. 15, 2013.

(51) Int. Cl. G01N 33/68; G01N 30/72; G01N 33/57505; H01J 49/00; H01J 49/16

(52) U.S. Cl. CPC G01N 33/6848; G01N 30/7233; G01N 33/57505; G01N 33/6851; G01N 33/6857; H01J 49/0027; H01J 49/164; G01N 2800/52

(57) ABSTRACT

Disclosure herein are methods for determining whether or not an immunoglobulin is present above the polyclonal background level in a biological sample, and methods for determining whether an immunoglobulin contains a kappa or lambda light chain. These methods are useful for screening biological samples for the presence or absence of monoclonal gammopathy, and for diagnosing and monitoring monoclonal gammopathy in a subject.

### CROSS-REFERENCE TO RELATED APPLICATIONS

This application is a continuation of U.S. application Ser. No. 14/777,236. Filed on Sep. 15, 2015, which is a National Stage application under 35 U.S.C. § 371 of International Application No. PCT/US2014/022475, having an International Filing Date of Mar. 10, 2014, claims the benefit of U.S. Provisional Application Ser. No. 61/792,944, filed Mar. 15, 2013. The disclosure of the prior applications are considered part of (and are incorporated by reference in) the disclosure of this application.

### TECHNICAL FIELD

This disclosure relates to methods and materials for identifying and quantifying a monoclonal immunoglobulin present above the polyclonal background in a sample, such as a biological sample.

### BACKGROUND

Human immunoglobulins contain two identical heavy chain polypeptides (each about 54 kilodaltons in MW) and two identical light chain polypeptides (each about 24 kilodaltons in molecular weight) which are bound together by disulfide bonds. Each light chain and each heavy chain include a constant region and a variable region. In healthy individuals, each plasma cell produces a single immunoglobulin having its own unique protein sequence contained within the variable regions of the fragment antigen binding (Fab) portion of the immunoglobulin. When examined in terms of molecular weight distribution, the mass spectrum of immunoglobulins or fragments containing the variable region(s) forms a normal distribution in a healthy individual. In a patient who has an abnormal expansion of a plasma cell, the abnormally expanded plasma cells all produce the same particular immunoglobulin, resulting in an overexpression of that immunoglobulin in the patient. A patient with such abnormality is at risk for developing serious diseases which collectively are known as monoclonal gammopathies.

The occurrence of a monoclonal immunoglobulin in the blood at a level above the normal immunoglobulin background can indicate a plasma B cell disorder. Serum protein gel electrophoresis (SPEP), immunofixation electrophoresis (IFE), urine protein gel electrophoresis (UPEP) and immunonephelometry are routine methods performed in clinical laboratories to confirm the presence of an abnormally high monoclonal immunoglobulin often referred to as an M-spike or M-protein-spike. The fundamental method of detection of these methods relies on either the differences in charge between immunoglobulins and interaction of specific antibodies with the immunoglobulins which are less specific properties than its mass.

There are two different isotypes of light chain polypeptides referred to as either kappa or lambda; and five different isotypes of heavy chain polypeptides referred to as gamma, alpha, mu, epsilon, and delta. Each of the two light chain polypeptides and each of the five heavy chain polypeptides contains two regions: the variable region and the constant region. The constant regions of the two types of light chains and five types of heavy chains have different amino acid sequences, and can be used to identify the isotype of the heavy or light chain. Current methods use antibody-based techniques to identify the isotype of the heavy or light chain. These antibodies are specific for each isotype only and hence do not directly detect clonality.

In certain diseases, such as monoclonal gammopathy, there is an increase in the amount of immunoglobulins in the bloodstream and in urine relative to a healthy individual. If high levels of immunoglobulin are detected, additional tests can be performed to determine the isotype of the light or heavy chain.

### SUMMARY

The present disclosure is based, at least in part, on the development of new mass spectrometry-based methods for determining whether or not a monoclonal immunoglobulin is present above the polyclonal background level, and in some embodiments for identifying and quantifying the same in a sample, and methods for determining whether the monoclonal immunoglobulin contains a kappa or lambda light chain; or a gamma, alpha, mu, epsilon, or delta heavy chain. The use of mass over charge ratio (m/z), optionally with the use of antibody interaction techniques, such as SPEP, IFE and immunoassays, provides a more direct assessment of the clonality because it measures a fundamental property of the monoclonal immunoglobulin, its mass. These methods are useful for screening biological samples for the presence or absence of an endogenous monoclonal immunoglobulin above the polyclonal background or an exogenous therapeutic monoclonal immunoglobulin, for monitoring the concentration of the monoclonal immunoglobulin in a subject, and for diagnosing and monitoring a monoclonal gammopathy.

In one aspect, this disclosure features mass spectrometry based methods for determining the presence or absence of, and optionally the identity and concentration of a monoclonal immunoglobulin above the polyclonal background level in a sample, e.g., a serum, plasma, whole blood, urine sample, or a man-made reagent solution. Immunoglobulins can be isolated from the sample and subjected to a mass spectrometry technique to determine whether or not an immunoglobulin is above the polyclonal background. The immunoglobulins can be isolated from the sample by chemical-based fractionation, e.g. Melon Gel chromatography, by affinity purification, e.g. Protein A, Protein G or Protein L purification, or by size exclusion chromatography.

In some embodiments, intact immunoglobulins can be subjected to the mass spectrometry assays described herein. In some embodiments, the immunoglobulins can be processed to reduce their total mass while retaining the unique variable regions of the immunoglobulins before subjecting to the mass spectrometry technique. In some cases, portions of immunoglobulins containing the variable regions are subjected to mass spectrometry. For example, the immunoglobulin light chains can be decoupled from the immunoglobulin heavy chains, and subjected to the mass spectrometry based methods disclosed herein. Any portion of the polypeptide chain or post-translational modification to the polypeptide chain can also be cleaved from the total immunoglobulin using proteases, and subjected to the mass spectrometry based methods disclosed herein.

In some embodiments, the mass spectrometry technique used in these methods can include a liquid chromatography mass spectrometry (LC-MS). In some embodiments, a matrix assisted laser desorption ionization mass spectrometry (MALDI MS) technique can be used. In some embodiments, tandem mass spectrometry (MS/MS) techniques can be used, for example, a liquid chromatography tandem mass spectrometry (LC-MS/MS) technique.

The mass spectrometry based methods disclosed herein, when coupled with a fast and effective immunoglobulin isolation method (e.g., Melon Gel Chromatography), can be used to screen patient samples, e.g. serum, plasma, whole blood, or urine samples, for endogenous monoclonal immunoglobulins present above the polyclonal background and/or exogenous therapeutic monoclonal immunoglobulins in a clinical laboratory setting. The mass spectrometry based screening methods disclosed herein show superior speed, sensitivity, resolution, and robustness than the conventional laboratory tests in screening for elevated concentration of a monoclonal immunoglobulin in biological samples.

In some embodiments, the immunoglobulin is a therapeutic monoclonal antibody. The mass spectrometry based methods disclosed herein further comprises determining the presence or absence of a therapeutic monoclonal antibody in the sample based on the presence or absence of a peak in the mass spectrum. In some embodiments, the quantification of the therapeutic monoclonal antibody in the sample can be determined based on the area of the peak of the mass of the variable region fragment.

In another aspect, the mass spectrometry-based methods disclosed herein can be used for diagnosing and monitoring, e.g., monitoring treatment of, progression of, remission of, etc., a monoclonal gammopathy in a subject. A sample can be obtained from the subject and subjected to mass spectrometry-based screening methods described above to provide a diagnosis of the presence or absence of the monoclonal gammopathy. When the subject is diagnosed to have monoclonal gammopathy, the methods disclosed herein can further be used to monitor a treatment of monoclonal gammopathy. Such methods include providing a first sample of the subject before the treatment and a second sample of the subject during or after the treatment. Immunoglobulins are isolated from the first and second samples, and subjected to a mass spectrometry technique. Level of an immunoglobulin present above the polyclonal background is determined before and after the treatment and compared. A decrease in its level indicates that the treatment may be effective for the subject; while an increase or no change in its level indicates that the treatment may be ineffective for the subject.

In some embodiments, the mass spectrometry based methods disclosed herein are combined with one or more conventional laboratory tests, for example, one or more of serum protein gel electrophoresis (SPEP), immunofixation electrophoresis (IFE), urine protein gel electrophoresis (UPEP) and immunonephelometry, to provide a thorough assessment of the clonality.

In yet another aspect, the disclosure features methods for determining whether an immunoglobulin light chain is a kappa or lambda light chain. Such methods can include fragmenting a preselected immunoglobulin light chain precursor ion using a tandem mass spectrometry technique to generate a distribution spectrum of fragment ions. The m/z's of one or more of the fragment ions can then be compared to m/z's of one or more fragment ions that are expected to result from the constant region of either kappa or lambda light chain. The immunoglobulin light chain precursor ion can be selected using a mass spectrometry technique.

As used herein, a “sample” can be any biological sample, such as a tissue (e.g., adipose, liver, kidney, heart, muscle, bone, or skin tissue) or biological fluid (e.g., blood, serum, plasma, urine, lachrymal fluid, or saliva) sample, or a man-made reagent.

As used herein, a “subject” is an animal such as a mammal, e.g. a human, dog, cat, primate, rodent, pig, sheep, cow, or horse.

As used herein, “variable region-containing immunoglobulins” can be intact immunoglobulins or portions of immunoglobulins containing the variable regions, e.g., immunoglobulin light chains, immunoglobulin heavy chains, antigen binding fragments (Fabs) of immunoglobulins, and mixtures thereof.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIGS. 1A-1D show results from the analysis of a serum sample from a patient with IgG kappa multiple myeloma. FIG. 1A is a mass spectrum showing a set of multiply charged ions that are converted to a molecular mass of 23 452.64 Da (FIG. 1B), which is within the expected mass range for an IgG light chain. FIG. 1C shows another set of multiply charged ions that are converted to a molecular mass of 51 596.07 and 51 758.27 Da (FIG. 1D), which is within the expected mass range for an IgG heavy chain. The difference between the molecular mass of series 2 and series 3 is 162.20 Da, which closely matches the mass of a hexose subunit.

FIGS. 2A-2D are a set of mass spectra of serum (FIGS. 2A and 2B) and urine samples (FIGS. 2C and 2D) from two patients one with a with an IgA kappa monoclonal gammopathy (FIGS. 2A and 2C) and one with an IgA lambda monoclonal gammopathy (FIGS. 2B and 2D), measured by LC-MS.

FIG. 3 is a light chain mass spectrum of a serum sample from a patient with an IgM monoclonal gammopathy, measured by LC-MS.

FIG. 4 is a light chain mass spectrum of a serum sample from a healthy individual without monoclonal gammopathy, measured by LC-MS.

FIG. 5 is a mass spectrum of the therapeutic monoclonal immunoglobulin adalimumab (HUMIRA®), measured by matrix assisted laser desorption ionization-time of flight mass spectrometry (MALDI-TOF MS) and LC-MS analysis.

FIG. 6 is a mass spectrum of a serum sample from a healthy individual without monoclonal gammopathy, measured by MALDI-TOF MS analysis.

FIG. 7 is a mass spectrum of a serum sample from a healthy individual spiked with diluted the therapeutic monoclonal immunoglobulin adalimumab, measured by MALDI-TOF MS analysis.

FIG. 8 is a mass spectrum of a serum sample from a patient with a lambda light chain multiple myeloma, measured by MALDI-TOF MS analysis.

FIG. 9 is a mass spectrum of a urine sample from a patient with a kappa free light chain, measured by MALDI-TOF MS analysis.

FIG. 10 is a mass spectrum of a serum sample from a patient with IgG kappa monoclonal gammopathy, measured by LC-MS, with highlights of the peak at 1229.9 Mass/Charge which is selected as the precursor ion for LC-MS/MS.

FIG. 11 shows the fragment ion mass spectrum for the precursor ion labeled in FIG. 10.

FIG. 12 shows the light chain mass spectra from different multiple myeloma patients with known kappa light chains.

FIG. 13 shows the light chain mass spectrum of a urine sample from a multiple myeloma patient with known lambda light chains along with the lambda light chain specific LC-MS/MS fragment ions.

FIG. 14 shows the light chain mass spectrum of a urine sample from a patient with known kappa free light chains, along with the kappa light chain specific LC-MS/MS fragment ions.

FIG. 15A-15B are a set of mass spectra from a normal serum sample (1A) and normal serum spiked with 0.5 g/dL of the IgG kappa recombinant mAb adalimumab (1B). The normal serum mass spectrum displays a broad range of unresolved peaks, whereas the normal serum spiked with 0.5 g/dL of adalimumab shows a clearly defined multiply charged protein ions. FIG. 15C is a converted spectrum for the normal serum sample displaying a broad range of unresolved peaks. FIG. 15D is a converted mass spectrum for the normal serum spiked with 0.5 g/dL of adalimumab showing a single peak at an average molecular mass of 23 412.19 Da. This mass is in excellent agreement with the calculated average molecular mass of 23 412.13 Da for the kappa light chain of adalimumab.

FIG. 16A-16C show results from the analysis of a serum sample from the same patient shown in FIG. 1 after treatment for multiple myeloma. The sample was negative by protein gel electrophoresis (PEL), immunofixation electrophoresis (IFE), and the free light chain (FLC) assay. FIG. 16A is a mass spectrum showing a series of multiply charged ions. FIG. 16B shows calculated molecular mass for the ions in FIG. A, which is only 0.47 Da different than the molecular mass observed in FIG. 1 for the light chain. FIG. 16A shows molecular mass for the proposed heavy chain ions can no longer be calculated because the ions are below the level of detection.

FIG. 17 shows results of the top-down MS of adalimumab spiked into normal serum. The ion at m/z=1233 in the top spectrum matches the +19 charge state ion from the kappa light chain of adalimumab and was selected for top-down MS. The arrow points to the fragment ion mass spectrum. The labeled fragment ions match the expected masses for fragment ions from the C-terminal portion of the kappa light chain that contains the constant region. The calculated y-ion masses for the kappa light chain constant region-specific amino acid sequence are shown in the table.

FIG. 18 shows results of the top-down MS of a lambda immunoglobulin light chain standard. The light chain ion at m/z=1193 in the top spectrum was selected for MS/MS, and the fragment ion mass spectrum is shown below. Fragment ions that match a portion of the lambda light chain constant region are labeled with their respective monoisotopic masses. The calculated b-ion monoisotopic masses for the lambda constant region-specific sequence are shown in the table.

Like reference symbols in the various drawings indicate like elements.

### DETAILED DESCRIPTION

The present disclosure is based, at least in part, on the development of new mass spectrometry-based methods for determining whether or not an immunoglobulin is present in a biological sample, and methods for determining whether the immunoglobulin contains a kappa or lambda light chain. The use of mass over charge (m/z), optionally with one or more antibody interaction techniques, such as SPEP, IFE and immunoassays, provides a more direct assessment of the clonality because it measures a fundamental property of the immunoglobulin. These methods are useful for screening biological samples for the presence or absence of monoclonal gammopathy, for diagnosing and monitoring monoclonal gammopathy in a subject, and for quantifying a monoclonal therapeutic antibody in a patient.

Described herein are methods based on mass spectroscopy to identify whether or not a monoclonal immunoglobulin is present in a biological sample. The speed, sensitivity, resolution, and robustness of mass spectroscopy make the present methods superior than SPEP or UPEP for screening samples for monoclonal gammopathies. A method described herein can include the use of a liquid chromatography mass spectrometry (LC-MS). In some embodiments, tandem mass spectrometry (MS/MS) techniques can be used, for example, a liquid chromatography tandem mass spectrometry (LC-MS/MS) technique. In some embodiments, matrix assisted laser desorption ionization-time of flight mass spectrometry (MALDI-TOF MS) technique can be used.

### Samples and Sample Preparation

A sample for analysis can be any biological sample, such as a tissue (e.g., adipose, liver, kidney, heart, muscle, bone, or skin tissue) or biological fluid (e.g., blood, serum, plasma, urine, lachrymal fluid, or saliva) sample, or a man-made reagent. The biological sample can be from a subject that has immunoglobulins, which includes but is not limited to a mammal, e.g. a human, dog, cat, primate, rodent, pig, sheep, cow, horse, bird, reptile, or fish.

A sample can be treated to remove components that could interfere with the mass spectrometry technique. A variety of techniques known to those having skill in the art can be used based on the sample type. Solid and/or tissue samples can be ground and extracted to free the analytes of interest from interfering components. In such cases, a sample can be centrifuged, filtered, and/or subjected to chromatographic techniques to remove interfering components (e.g., cells or tissue fragments). In yet other cases, reagents known to precipitate or bind the interfering components can be added. For example, whole blood samples can be treated using conventional clotting techniques to remove red and white blood cells and platelets. A sample can be deproteinized. For example, a plasma sample can have serum proteins precipitated using conventional reagents such as acetonitrile, KOH, NaOH, or others known to those having ordinary skill in the art, optionally followed by centrifugation of the sample.

Immunoglobulins can be isolated from the samples using standard methods known in the art. For example, the immunoglobulins can be purified by chemical-based fractionation, e.g., Melon Gel Chromatography (Thermo Scientific), where Melon Gel resins bind to non-immunoglobulin proteins in a sample and allow immunoglobulins to be collected in the flow-through fraction; or by affinity purification, e.g., by Protein A, Protein G, or Protein L purification, where immunoglobulins are bound by those proteins at physiologic pH and then released from the proteins by lowering the pH. When serum, plasma, or whole blood samples are used, a sample, such as a 10-250 μl sample, e.g., a 50 μl, can be directly subjected to Melon Gel, Protein A, Protein G, or Protein L purification. When urine samples are used, a urine sample can be buffered, e.g., a 50 μl urine sample can be diluted first with 50 μl of 50 mM ammonium bicarbonate.

Intact immunoglobulins can be further processed to reduce their overall mass while retaining the unique variable region of the immunoglobulin. In some embodiments, the light chains in a total immunoglobulin sample can be decoupled from the heavy chain immunoglobulins. Decoupling can be achieved by treating the total immunoglobulins with a reducing agent, such as dithiothreitol, tris(2-carboxyethyl) phosphine, or 2-mercaptoethanol. In some embodiments, the reducing step is performed at elevated temperature, e.g., in a range from about 30° C. to about 65° C., such as about 55° C., in order to denature the proteins. In some embodiments, the sample is further treated, e.g., by modifying the pH of the sample or buffering the sample. In some embodiments, the sample can be acidified.

In some embodiments, the antigen binding fragments (Fab) of immunoglobulins can be cleaved from the intact immunoglobulins using proteases such as pepsin. Excess reagents and salts can be removed from the samples using methods known to those having ordinary skill in the art.

### Mass Spectrometry Methods

After sample preparation, an immunoglobulin sample, such as an intact, decoupled light chain or Fab immunoglobulin sample, can be subjected to a mass spectrometry (MS) technique, either directly or after separation on a high performance liquid chromatography column (HPLC). In some embodiments, liquid chromatography mass spectrometry (LC-MS) can be used to analyze the mass spectrum of the ions, e.g., the +1 ions, resulting from the intact, light chain or Fab immunoglobulin sample. LC-MS is an analytical technique that combines the physical separation capabilities of liquid chromatography with the mass analysis capabilities of mass spectrometry, and is suitable for detection and potential identification of chemicals in a complex mixture. Any LC-MS machine can be used, e.g., the ABSciex 5600 Mass Spectrometer. The ion mass spectrum can be analyzed for one or more peaks having an intensity greater than the intensity of the background ion levels, e.g., the ions resulting from non-overexpressed immunoglobulins. For example, one or more ion peaks, e.g., an ion peak of the highest intensity, can be examined to determine if the one or more ion peaks has an ion intensity greater than the background intensity. In some embodiments, the ion intensity of the one or more peaks is at least two standard deviations greater than the background intensity; in some cases, at least 50% greater, at least 75% greater, or at least 100% greater, or at least 3-fold higher, 5-fold higher, 6-fold, 7-fold, 8-fold, 9-fold, 10-fold higher, 25-fold higher, 50-fold higher, 75-fold higher, 100-fold higher, or more. The presence of one or more peaks having an ion intensity greater than the background level is considered as an M-protein peak or M-spike, indicating the presence of a monoclonal immunoglobulin above the polyclonal background.

In some embodiments, matrix assisted laser desorption ionization-time of flight mass spectrometry (MALDI-TOF MS) can be used to analyze the mass spectrum of an immunoglobulin sample, e.g., the mass spectrum of the +1 charge state of the molecules in the sample, i.e., the intact, light chain or Fab immunoglobulin sample. Matrix-assisted laser desorption/ionization mass spectrometry (MALDI MS) uses a soft ionization technique to obtain large ions in the gas phase, and is suitable for analyzing fragile biomolecules (such as DNA, proteins, peptides and sugars) and large organic molecules, which tend to be fragmented when ionized by conventional ionization methods. The time-of-flight (TOF) analyzer uses an electric field to accelerate the ions through the same potential, and then measures the time they take to reach the detector. If the particles all have the same charge, the kinetic energies are identical, and their velocities depend only on their masses. Lighter ions reach the detector first. Samples can be prepared using a dried droplet method for MALDI-TOF MS. The advantages of using MALDI-TOF MS include: 1) lower instrument costs, 2) higher throughput, 3) easy sample preparation, 4) easy to use instrumentation, and 5) lower charge states. Any MALDI-TOF mass spectrometer can be used, e.g., the Biflex III MALDI-TOF Mass Spectrometer (Bruker Daltonics). The mass spectrum, e.g., the mass spectrum of +1 intact light chain polypeptide ions, can be analyzed to identify one or more peaks having an ion intensity greater than the background ion intensity and at an appropriate mass/charge expected for a light chain or Fab immunoglobulin fragment, e.g., about 21,000 to about 26,000 m/z, or about 22,000 to about 24,500 m/z, or about 23,000 to about 24,000 m/z for light chains; or about 40,000 to about 65,000 m/z, or about 45,000 to about 62,000 m/z; or about 50,000 to about 60,000 m/z for Fab immunoglobulin fragments. In some embodiments, the one or more peaks has an ion intensity at least two standard deviations greater than background ion intensity; or in some embodiments, at least 50% greater, at least 75% greater, or at least 100% greater, or at least 3-fold higher, 5-fold higher, 6-fold, 7-fold, 8-fold, 9-fold, 10-fold higher, 25-fold higher, 50-fold higher, 75-fold higher, 100-fold higher, or more, than the background ion intensity. The presence of one or more peaks having an ion intensity greater than the background ion intensity level and at an appropriate m/z for a light chain or Fab immunoglobulin fragment is considered as an M-protein peak or M-spike, indicating the presence of a monoclonal immunoglobulin above the polyclonal background.

In some embodiments, tandem mass spectrometry (MS/MS) can be used to determine whether an immunoglobulin contains a kappa light chain. For example, two rounds of MS can be performed. During the first round, the sample, e.g., a decoupled immunoglobulin light chain sample, is subjected to LC-MS, and a light chain ion fragment mass spectrum is generated, e.g., a distribution of light chain immunoglobulin fragments having +1 m/z's, as described above. The most intense ion peak is identified and selected as the precursor ion for the second round of mass spectrometry, LC-MS/MS. Once the m/z of the precursor ion of highest intensity is determined, the LC-MS/MS method allows the quadrupole portion of the mass spectrometer to select for that specific ion. During the second round of LC-MS/MS, this precursor ion is fragmented using collision-induced dissociation (CID), which involves the collision of an ion with a neutral atom or molecule in the gas phase and subsequent dissociation of the ion. The fragment ions produced during CID can then be detected using the time-of-flight (TOF) portion of the mass spectrometer. One or more of the m/z's (e.g., one, two, three, four, five, six, seven, eight or more) of the resulting distribution of fragment ion peaks can be compared to a list of one or more expected m/z's for protein fragment ions, e.g., protein fragment +1 ions, that would be expected to result from a light chain's C-terminal constant region. Light chain constant region amino assay sequence is available on public databases. Such ions are referred to as y-ions for the C-terminal constant region of the kappa light chain. When one or more of the fragment ions m/z's correlate, e.g., match, with one or more of the m/z's of the expected protein fragment ions, e.g., one, two, three, four, five, size, seven, eight, or more, the immunoglobulin light chain is determined to be a kappa light chain.

Similarly, tandem mass spectrometry can be used to determine whether an immunoglobulin contains a lambda light chain. For example, two rounds of MS can be performed, and during the first round, the sample, e.g., a decoupled immunoglobulin light chain sample, is subjected to LC-MS, and a light chain ion fragment mass spectrum is generated, e.g., a distribution of light chain immunoglobulin fragments having +1 m/z's, as described above. A precursor ion is selected for the second round LC-MS/MS. Preferably, but not necessarily, the precursor ion is selected from the most abundant ion within the mass range for precursor ion selection. Once the m/z of the precursor ion is determined, the LC-MS/MS method allows the quadrupole portion of the mass spectrometer to select for that specific ion. During the second round of LC-MS/MS, this precursor ion is fragmented using collision-induced dissociation (CID), which involves the collision of an ion with a neutral atom or molecule in the gas phase and subsequent dissociation of the ion. The fragment ions produced during CID can then be detected using the time-of-flight (TOF) portion of the mass spectrometer. One or more of the m/z's (e.g., one, two, three, four, five, six, seven, eight or more) of the resulting distribution of fragment ion peaks can be compared to a list of one or more expected m/z's for protein fragment ions, e.g., protein fragment +1 ions, that would be expected to result from a light chain's N-terminal portion of the constant region. Such ions are referred to as b-ions for the N-terminal portion of lambda constant region. The light chain constant region amino assay sequence is available on public databases. The comparison can be performed using commercially available software, e.g. ProSight PTM 2.0. When one or more of the fragment ions m/z's correlate, e.g., match, with one or more of the m/z's of the expected protein fragment ions, e.g., one, two, three, four, five, size, seven, eight, or more, the immunoglobulin light chain is determined to be a lambda light chain.

### Methods for Screening Biological Samples, and for Diagnosing and Monitoring Monoclonal Gammopathy

The mass spectrometry based methods disclosed herein can be used to determine whether or not a particular immunoglobulin is present in a biological sample. Immunoglobulins can be isolated from the biological sample and subjected to a mass spectrometry technique to determine whether or not an immunoglobulin is present above the polyclonal background. In some embodiments, intact immunoglobulins can be subjected to the mass spectrometry assays. In some embodiments, the immunoglobulins can be processed to reduce their mass while retaining the unique variable regions of the immunoglobulins before subjecting to the mass spectrometry technique. In those cases, portions of immunoglobulins containing the variable regions are subjected to mass spectrometry. For example, the immunoglobulin light chains can be decoupled from the immunoglobulin heavy chains, and subjected to the mass spectrometry based methods disclosed herein. The antigen binding fragments (Fab) of immunoglobulins can also be cleaved from the total immunoglobulin using enzymes such as pepsin, and subjected to the mass spectrometry based methods disclosed herein.

The mass spectrometry based methods disclosed herein when coupled with a fast and effective immunoglobulin isolation method (e.g., Melon Gel Chromatography) can be used to screen patient samples, e.g. serum, plasma, whole blood, or urine samples, for monoclonal gammopathies in a clinical laboratory setting. The mass spectrometry based screening methods disclosed herein show superior speed, sensitivity, resolution, and robustness than the conventional laboratory tests in screening for elevated monoclonal immunoglobulin expression in biological samples.

The mass spectrometry based methods disclosed herein can be used for diagnosing and monitoring monoclonal gammopathy in a subject. A sample can be obtained from the subject and subject to mass spectrometry based screening methods described above to provide a diagnosis of the presence or absence of the monoclonal gammopathy. When the subject is diagnosed to have monoclonal gammopathy, the methods disclosed herein can further be used to monitor a treatment of monoclonal gammopathy. Such methods include providing a first sample of the subject before the treatment and a second sample of the subject during or after the treatment. Immunoglobulins are isolated from the first and second samples, and subjected to a mass spectrometry technique. Level of an immunoglobulin is determined before and after the treatment and compared. A decrease in its level indicates that the treatment may be effective for the subject; while an increase or no change in its level indicates that the treatment may be ineffective for the subject.

### Methods for Determining Whether an Immunoglobulin Contains a Kappa or Lambda Light Chain

Knowing the type of light chain, either kappa or lambda, and its quantity associated with the M-spike, are critical for monoclonal gammopathy diagnosis and prognosis. Clinical labs commonly use immunofixation electrophoresis to identify the type of immunoglobulin light chains and use agarose gel electrophoresis to quantify them.

The methods described herein are based on the discovery of a unique top down fragmentation pattern produced by intact kappa and lambda light chains. Since kappa and lambda light chains each have unique C-terminal amino acid sequences, tandem mass spectrometry (MS/MS) on the intact ions can be performed to determine whether an immunoglobulin light chain is kappa or lambda light chain. Intact kappa ions when exposed to collision-induced dissociation (CID) during the MS/MS produce y-ion fragments from the C-terminal portion of the kappa constant region. Intact lambda ions when exposed to the same CID condition during MS/MS produce b-ions from the N-terminal portion of the lambda constant region.

Based on these findings, tandem mass spectrometry based methods for determining whether an immunoglobulin light chain is a kappa or lambda light chain have been developed. Such methods can include the steps of: (1) fragmenting a preselected immunoglobulin light chain precursor ion using a tandem mass spectrometry technique to generate a distribution spectrum of fragment ions; and (2) comparing the m/z's of one or more of the fragment ions to m/z's of one or more fragment ions that are expected to result from the constant region of either kappa or lambda light chain. The immunoglobulin light chain precursor ion can be selected using a mass spectrometry technique.

### EXAMPLES

The invention is further described in the following examples, which do not limit the scope of the invention described in the claims.

### Example 1. Detection of Monoclonal Immunoglobulins Using LC-MS Based Methods in Serum Samples from Patients with Documented IgG, IgA, or IgM Monoclonal Gammopathy

Total immunoglobulins were isolated by subjecting a 50 μl serum sample from a subject to Melon Gel (Thermo Scientific) purification according to manufactures insert. The light chain immunoglobulins were decoupled from heavy chain immunoglobulins by reducing and denaturing the total immunoglobulins with 50 mM dithiothreitol (DTT) for 1 hour at 55° C. The decoupled immunoglobulin sample was diluted in water and excess reagents were removed using a 3 kDa filter tube. The decoupled immunoglobulin sample was acidified with 0.1% formic acid, and then examined by liquid chromatography-mass spectrometry (LC-MS) using ABSciex 5600 mass spectrometer equipped with a C8 reverse phase column.

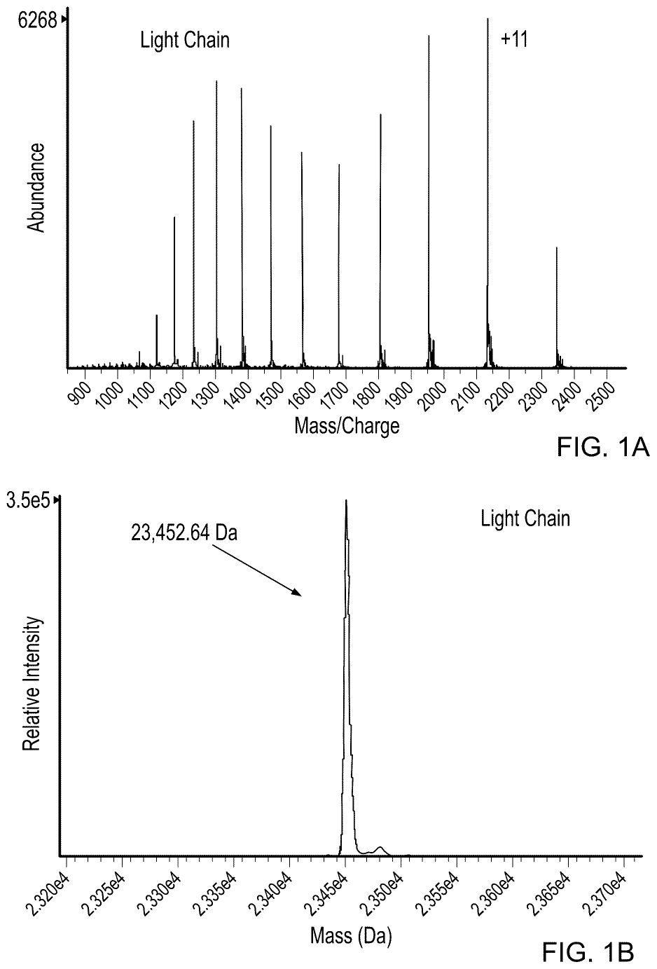

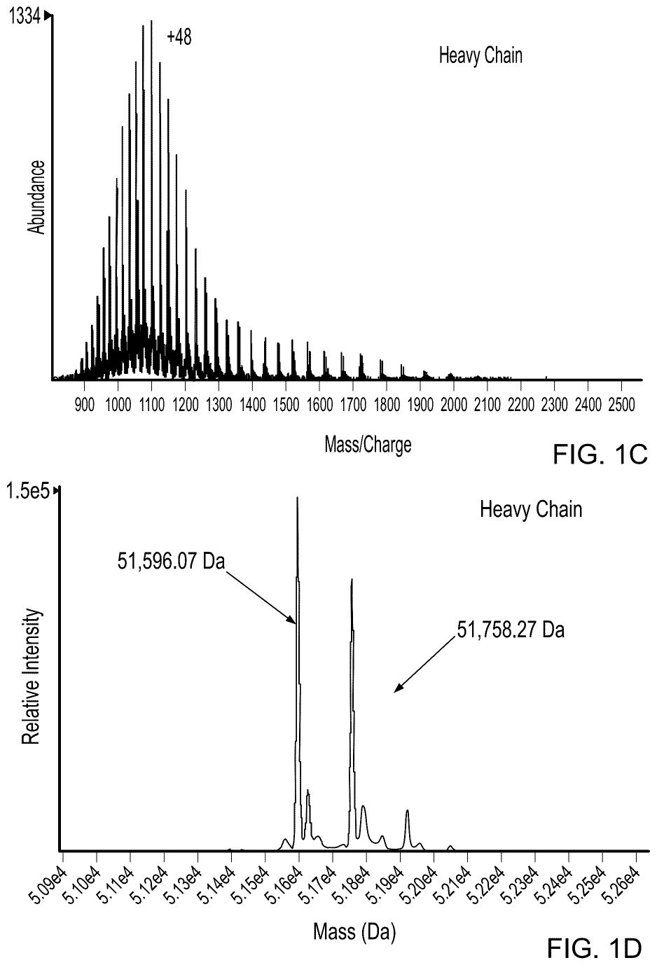

FIGS. 1A-1D show results from the analysis of a serum sample from a patient with IgG kappa multiple myeloma using methods described herein. FIG. 1A is a mass spectrum showing a set of multiply charged ions that are converted to a molecular mass of 23 452.64 Da (FIG. 1B), which is within the expected mass range for an IgG light chain. FIG. 1C shows another set of multiply charged ions that are converted to a molecular mass of 51 596.07 and 51 758.27 Da (FIG. 1D), which is within the expected mass range for an IgG heavy chain. The difference between the molecular mass of series 2 and series 3 is 162.20 Da, which closely matches the mass of a hexose subunit.

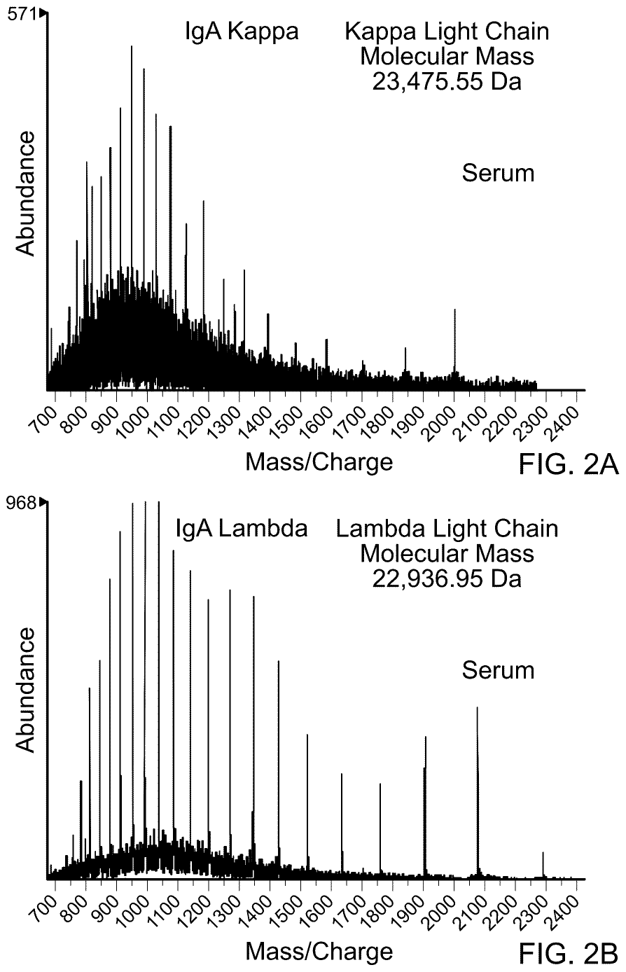

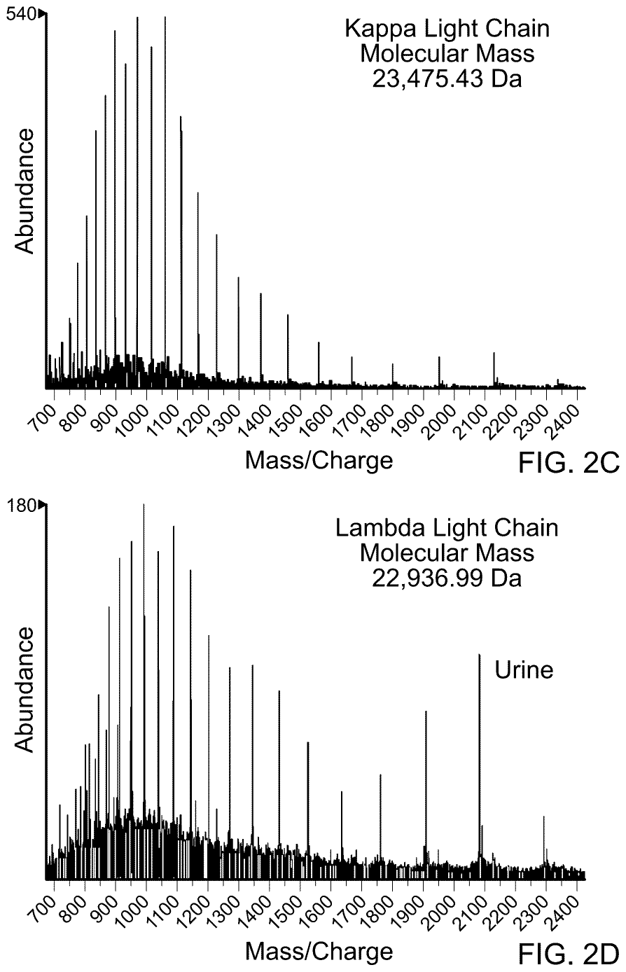

FIGS. 2A-2D show mass spectra of the serum (FIGS. 2A and 2B) and urine samples (FIGS. 2C and 2D) from two patients one with a with an IgA kappa monoclonal gammopathy (FIGS. 2A and 2C) and one with an IgA lambda monoclonal gammopathy (FIGS. 2B and 2D), measured by LC-MS.

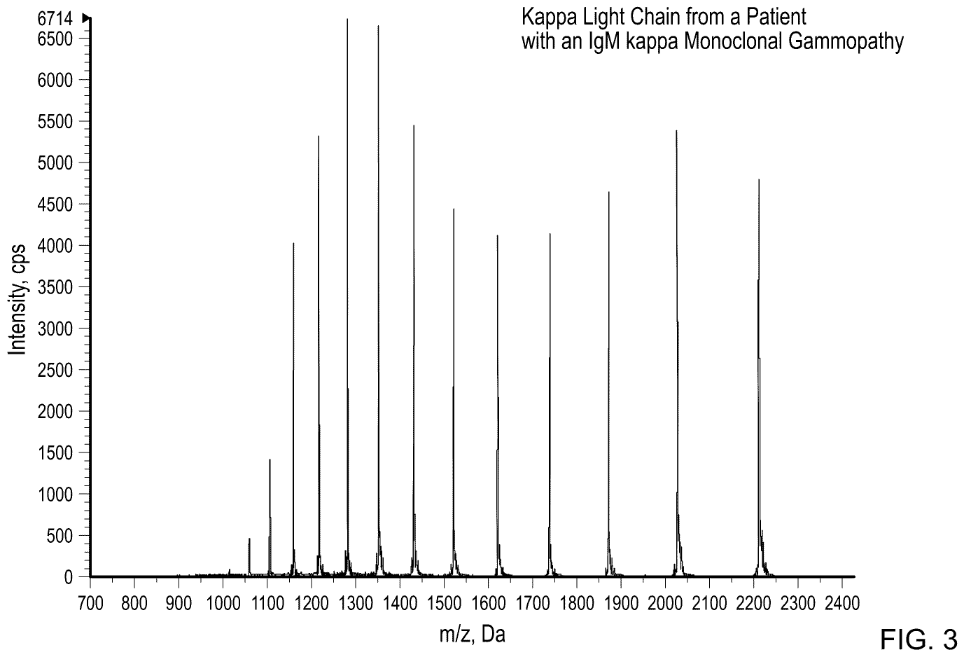

FIG. 3 shows a light chain mass spectrum of a serum sample from a patient with an IgM monoclonal gammopathy, measured by LC-MS.

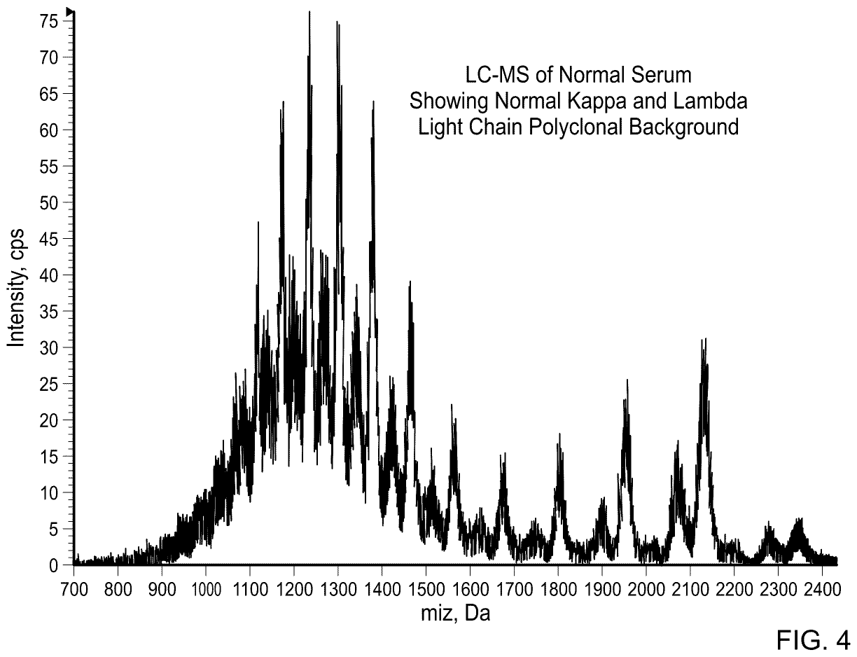

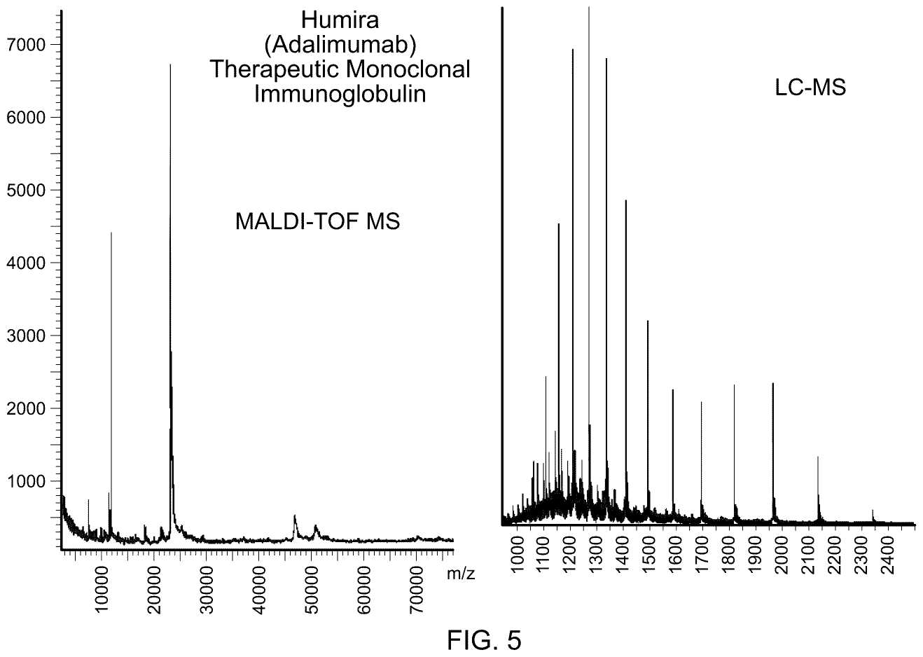

FIG. 4 shows a light chain mass spectrum of a serum sample from a healthy individual without monoclonal gammopathy, measured by LC-MS.

### Example 2. Detection of Immunoglobulin Light Chains in Serum or Urine Samples by MALDI-TOF MS

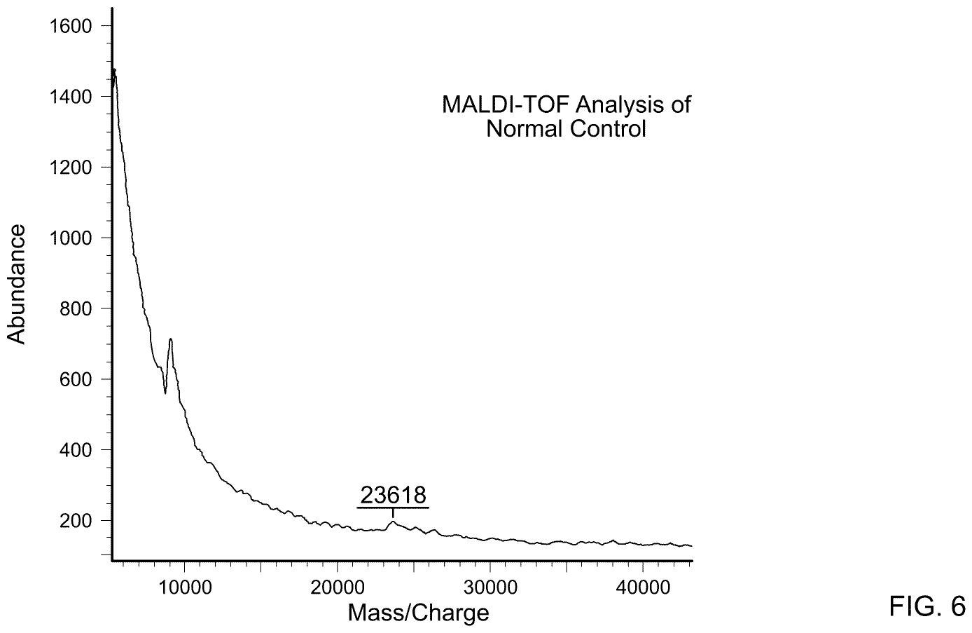

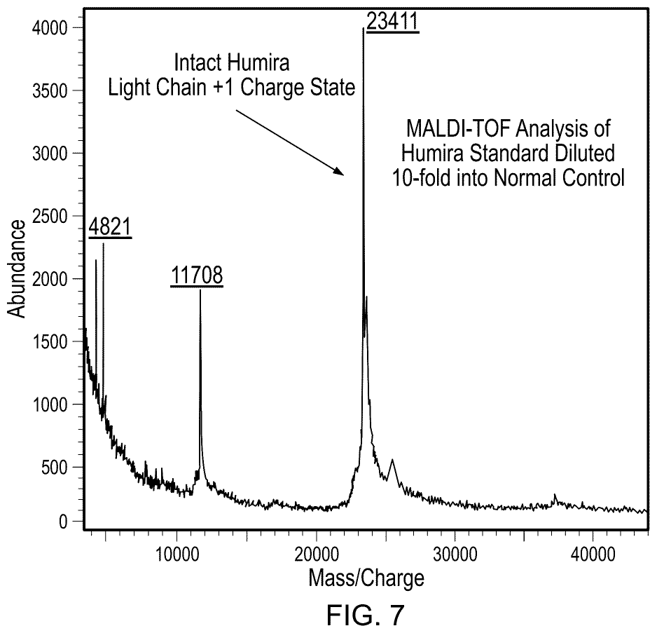

Matrix Assisted Laser Desorption Ionization-Time of Flight Mass Spectrometry (MALDI-TOF MS) was also used for monitoring monoclonal antibodies in serum and urine. Melon Gel purified adalimumab (HUMIRA®) and normal serum controls were used to get initial results using MALDI-TOF MS (FIGS. 5-7). The most abundant peak representing the immunoglobulin light chain in FIG. 5 is roughly 3000 counts for the adalimumab, while the background level of immunoglobulin light chains in the normal control sample in FIG. 6 is roughly 50 counts. Next, the adalimumab was diluted tenfold and spiked into a normal control serum sample to simulate the presence of a monoclonal immunoglobulin in a patient serum sample and analyzed by MALDI-TOF MS. As shown in FIG. 7, adalimumab was detected in the normal serum sample by MALDI-TOF MS.

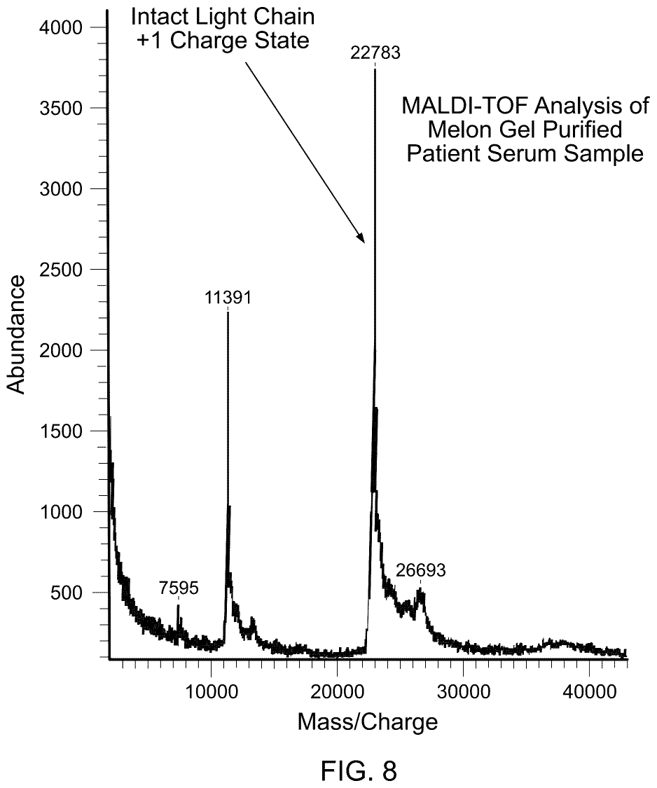

MALDI-TOF MS was then used to identify monoclonal antibodies in serum samples from a patient with multiple myeloma. Serum sample was prepared as describe above in Example 1 and analyzed by MALDI-TOF MS. The monoclonal light chain antibody associated with the malignant clone was clearly seen at Mass/Charge 22,783 (FIG. 8).

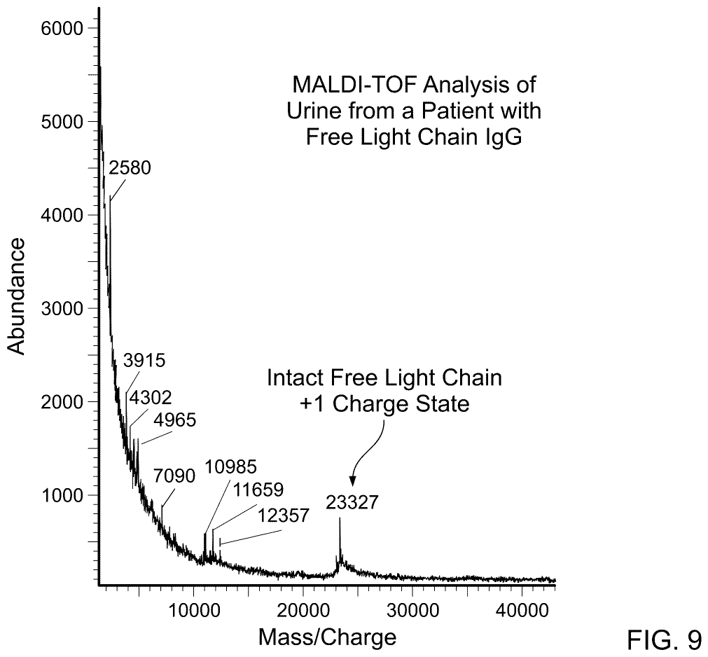

The ability of MALDI-TOF MS to identify the presence of monoclonal free light chains (>3 mg/mL) in urine sample from patients with multiple myeloma was also evaluated. A 50 μl urine sample from the patient was diluted first with 50 μl of 50 mM ammonium bicarbonate and then reduced with 100 mM DTT for 30 minutes at 55° C. The reduced sample was acidified with formic acid then examined by MALDI-TOF MS. The results from the MALDI-TOF MS analysis of a urine sample from a patient with known lambda free light chains were shown in FIG. 9, and a peak at 23,327 Mass/Charge indicates the presence of the lambda free light chain in the urine sample. These findings indicate that MALDI-TOF MS can be used to screen patient samples for monoclonal gammopathies.

### Example 3. Determination of Whether an Immunoglobulin Light Chain is Lambda or Kappa Light Chain by Tandem Mass Spectrometry

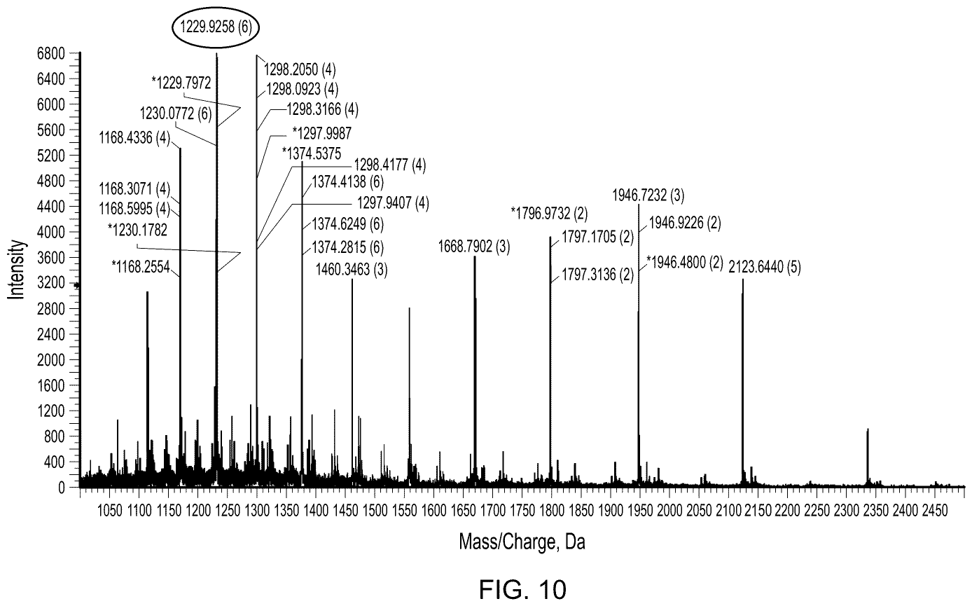

FIG. 10 is a mass spectrum of a serum sample from a patient with IgG kappa monoclonal gammopathy, measured by LC-MS, with highlights of the peak at 1229.9 Mass/Charge which is selected as the precursor ion for LC-MS/MS. Once the precursor ion mass was determined, an LC-MS/MS method was selected that allows the quadrupole portion of the mass spectrometer to select that specific ion. This precursor ion was then fragmented using collision-induced dissociation (CID) in the collision cell portion of the mass spectrometer. The fragment ions produced were then analyzed using the time-of-flight (TOF) portion of the mass spectrometer.

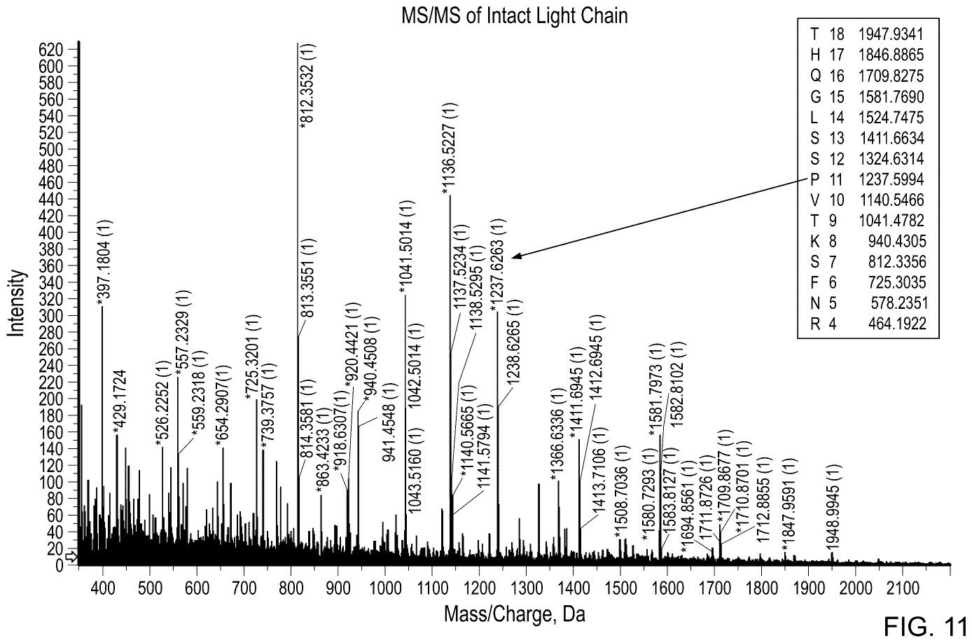

FIG. 11 showed the fragment ion spectrum for the precursor ion labeled in FIG. 10. On the right hand side of FIG. 11 was a list of C-terminal y-ion fragments and their masses from the constant region for kappa light chain. FIG. 11 also showed an arrow pointing from the list at P (proline) residue 11 with a mass of 1237.5994, to a peak in the fragment ion spectrum at mass 1237.6263. By comparing other fragment ion peaks with the list of y-ions for the C-terminal constant region for kappa light chain, it was possible to identify the M-spike as a kappa light chain.

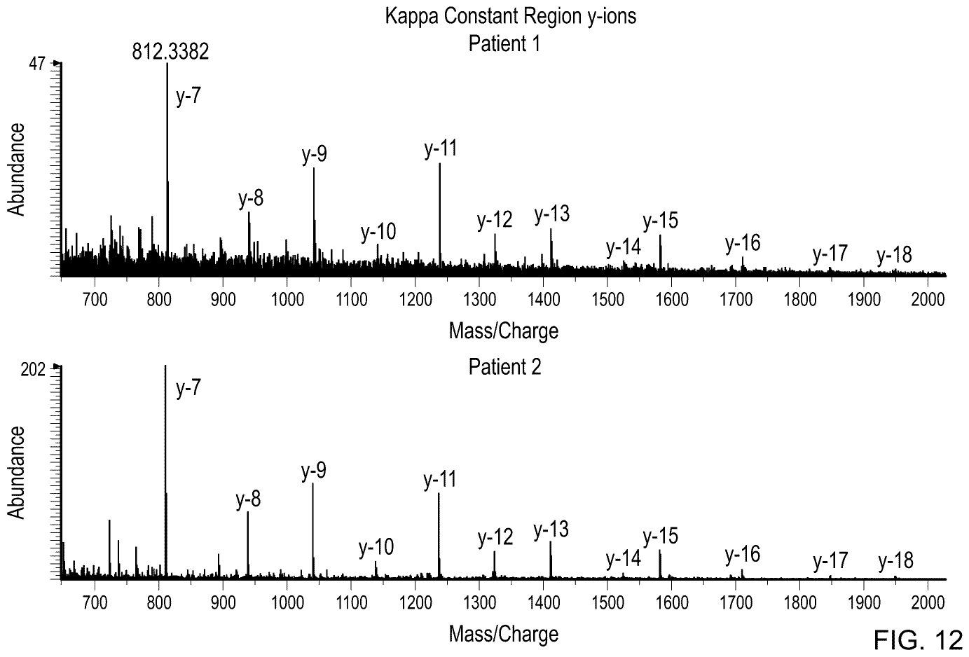

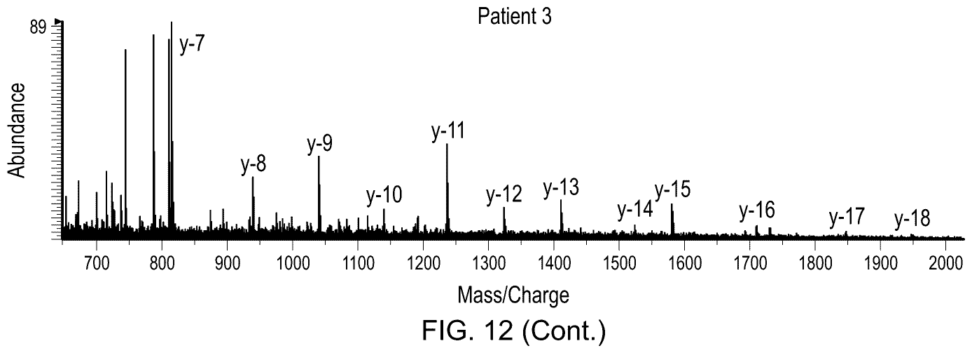

Serum sample from several multiple myeloma patients with known M-spike kappa light chains, were prepared as described above in Example 1. FIG. 12 showed the M-spike light chain mass spectra of the serum samples from different multiple myeloma patients with known M-spike kappa light chains. Each spectrum showed a different set of multiply charged ions from each patient's unique light chain. These results clearly showed that LC-MS/MS of the intact light chain can be used to determine if it is a kappa light chain.

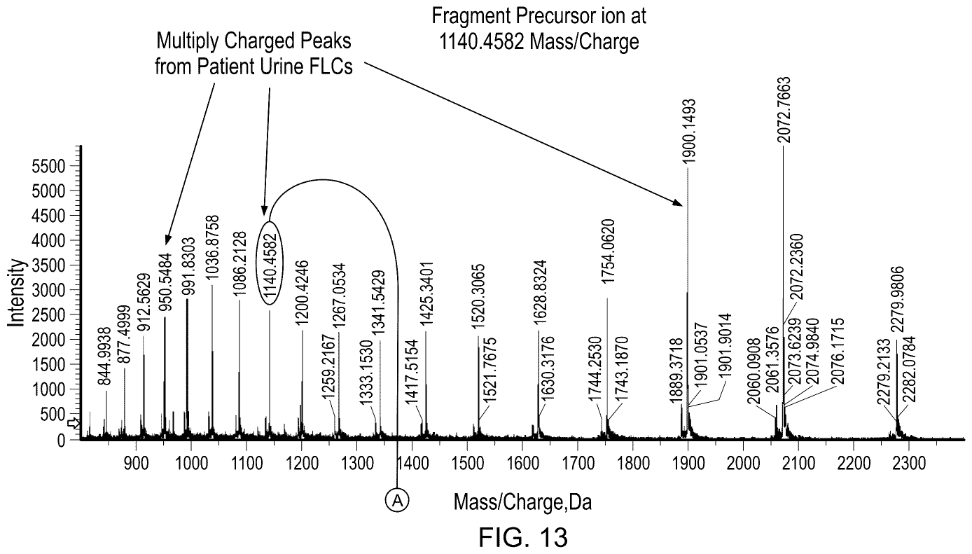

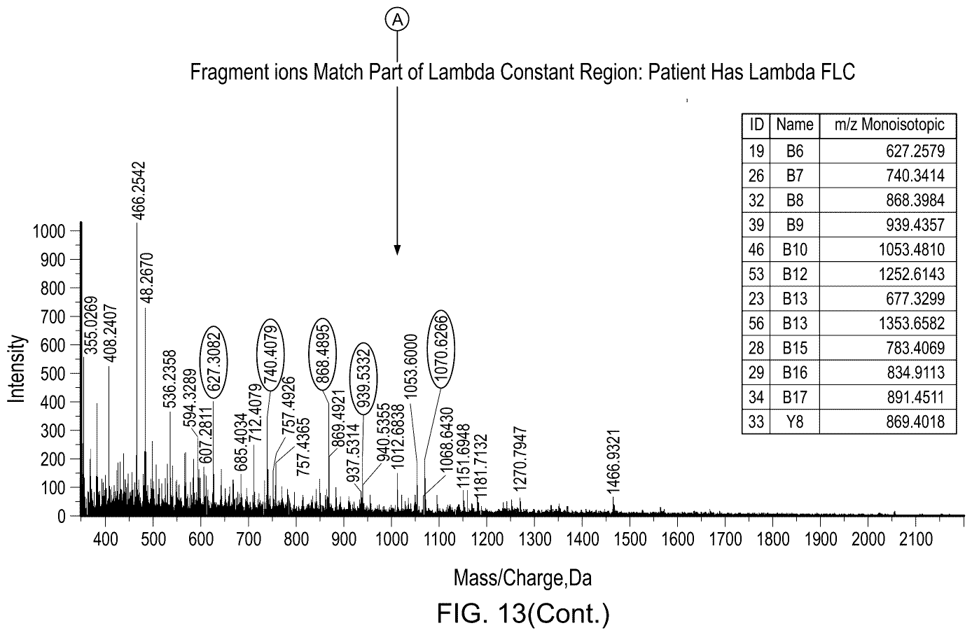

Equivalent experiments were performed on multiple myeloma patients with known M-spike lambda light chains found in urine. Urine samples were prepared as described in Example 1. Data obtained for patient urine samples demonstrated the superior analytical sensitivity and specificity of the method over methods currently used in clinical laboratories, for example serum protein gel electrophoresis (SPEP), or urine protein gel electrophoresis (UPEP). FIG. 13 showed the M-spike light chain mass spectrum from a patient urine sample with known M-spike lambda light chains. The spectrum at the top of FIG. 13 showed the multiply charged ions from lambda free light chains (FLCs) found to be present in the patient's urine using a standard immunoassay. The spectrum at the bottom of FIG. 13 showed the LC-MS/MS fragment ion spectra from the patient's intact lambda light chain ion at 1140.4582 Mass/Charge. By using a commercially available software package called Prosight, the identity of the fragment ions observed can be determined. The software identified the ions as b-ions produced from the N-terminal portion of the lambda constant region. Ions that match the expected b-ions were circled in the lower spectrum in FIG. 13. These results clearly showed that LC-MS/MS of an intact light chain can be used to determine if it is lambda light chain.

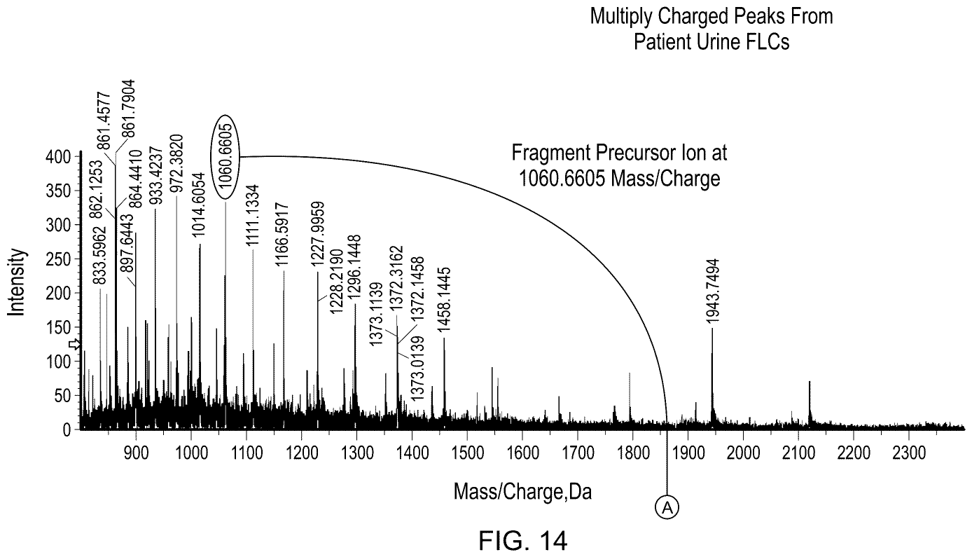

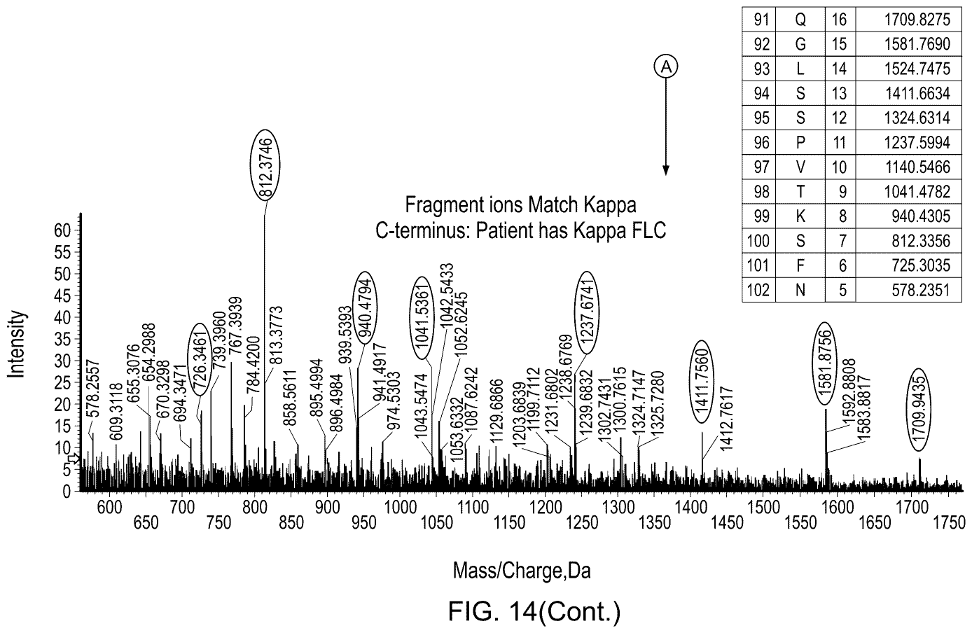

Urine samples from patient with known kappa free light chains were tested using the same method. The results from these experiments were shown in FIG. 14. The top of FIG. 14 showed the spectrum for the intact kappa free light chain in the urine sample. The spectrum was similar to those observed for serum. The bottom of FIG. 14 showed the fragment ion spectrum for the intact multiply charged ion at 1060.6605 Mass/Charge. Fragment ions that match the expected ions from the constant region of the C-terminus were highlighted. The fragment ions observed were the same as those observed in serum samples from patients with a kappa light chain (see FIG. 11). This observation confirmed that kappa light chains can be identified in urine and serum samples.

Clear differences in the fragment ions were produced between kappa and lambda light chains and can be detected by tandem mass spectrometry. Intact kappa ions exposed to collision-induced dissociation (CID) during the MS/MS portion of the experiment on the 5600 Q-TOF mass spectrometer had produced y-ion fragments from the C-terminal portion of the molecule. Intact lambda ions exposed to the same CID, MS/MS conditions had produced b-ions from the N-terminal portion of the molecule. Similar method can be used to identify the different lambda subtypes giving an additional diagnostic aspect to this methodology. The unique y-ion series produced by the C-terminal amino acid sequence from kappa can be used as an MS/MS tag similar to protein tags that are added to recombinant proteins for purification purposes.

### Example 4. Adalimumab in Normal Serum as a Model System

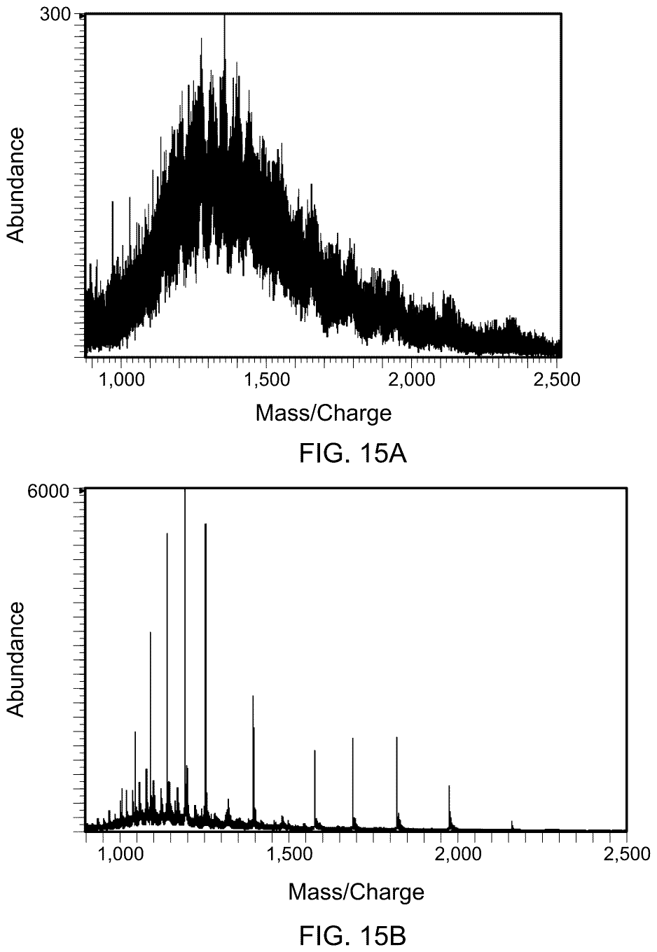

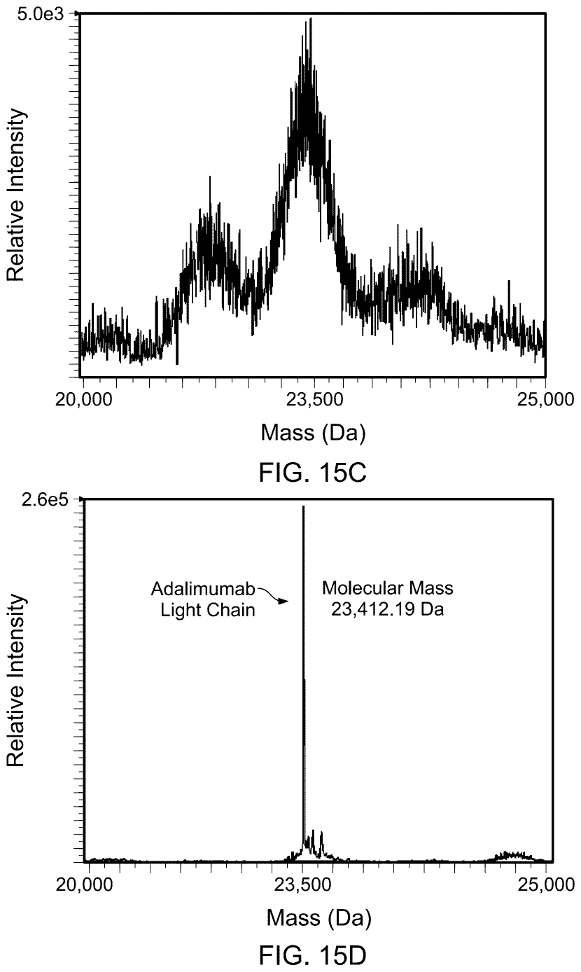

Adalimumab is an anti-TNF therapeutic monoclonal immunoglobulin that is widely prescribed for downregulating the inflammatory response in patients with autoimmune disorders. Therapeutic monoclonal immunoglobulins such as adalimumab are ideal surrogate standards for simulating a monoclonal immunoglobulin in serum because they are readily available in high purity and typically have a large body of literature on their structural properties. FIG. 15 shows the mass spectra for normal serum and serum spiked with 0.5 g/dL (30 μM) of adalimumab. Each mass spectrum represents the spectra summed together over the adalimumab light chain elution time. The mass spectrum from normal serum in section A shows a broad unresolved peak with a maximum relative abundance of 300 counts per second (cps). Alternatively the mass spectrum from the serum spiked with adalimumab in section B shows a distinct series of peaks from multiply charged protein ions with a maximum relative abundance of 6000 cps. The converted molecular mass for the normal serum in panel C shows a set of broad distribution of masses with no single mass higher in abundance than the background. This is in sharp contrast to the converted molecular mass for the normal serum spiked with 0.5 g/dL of adalimumab in panel D, which displays a single peak with the observed molecular mass of 23 412.19 Da. This molecular mass agrees with the average molecular mass calculated from the known amino acid sequence for the kappa light chain of adalimumab (23 412.13 Da). To assess microLC-ESI-Q-TOF MS for quantification, adalimumab was spiked into 50 mM ammonium bicarbonate buffer, normal serum, and normal urine. Ten different standard concentrations were used ranging from 0.005 to 5.0 g/dL. Standard curves made in serum used Melon Gel to enrich for immunoglobulins, whereas curves made in urine and buffer were reduced and analyzed without Melon Gel purification. Linearity and linear dynamic range values in the table are split according to the two quantification techniques. The first approach labeled “deconvolution peak area” uses the peak area found after deconvolution of the multiply charged ions to molecular mass, whereas the second approach labeled “extracted ion peak area” refers to using the peak areas obtained from a selected set of extracted ions. The table demonstrates that the standard curves have a linear dynamic range within the concentration range needed in clinical practice. The interassay precision of 10 replicate Melon Gel preparations of adalimumab spiked into normal serum at 0.1 g/dL was examined and found the CV for the peak area of the light chain to be 6.2%, whereas the CV for the heavy chain was 11%. The limit of quantification as defined by a CV<20% for 10 replicates using the deconvolution peak areas was 0.005 g/dL for the light chain and 0.025 g/dL for the heavy chain of adalimumab spiked into normal serum.

### Example 5. Monitoring a Monoclonal Immunoglobulin in a Patient with Multiple Myeloma

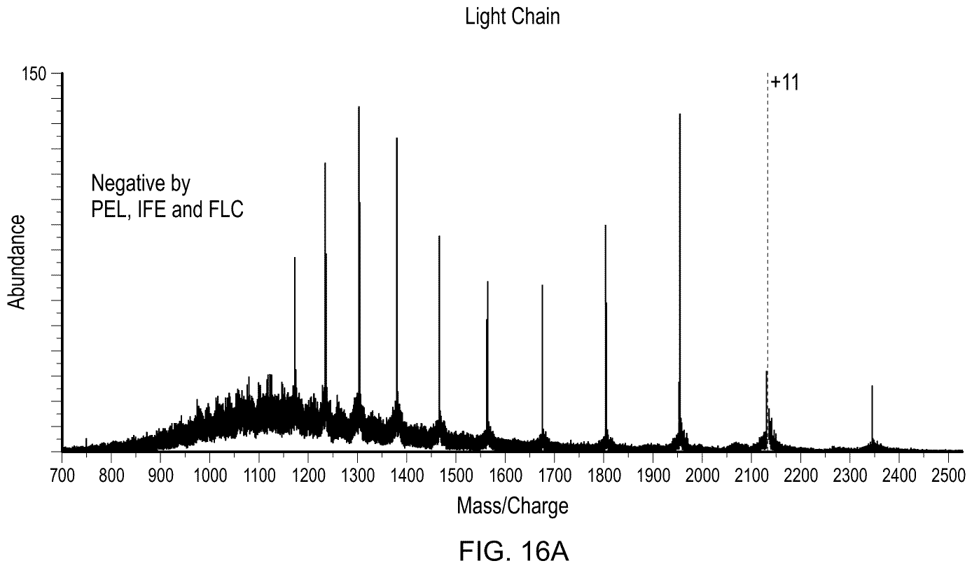

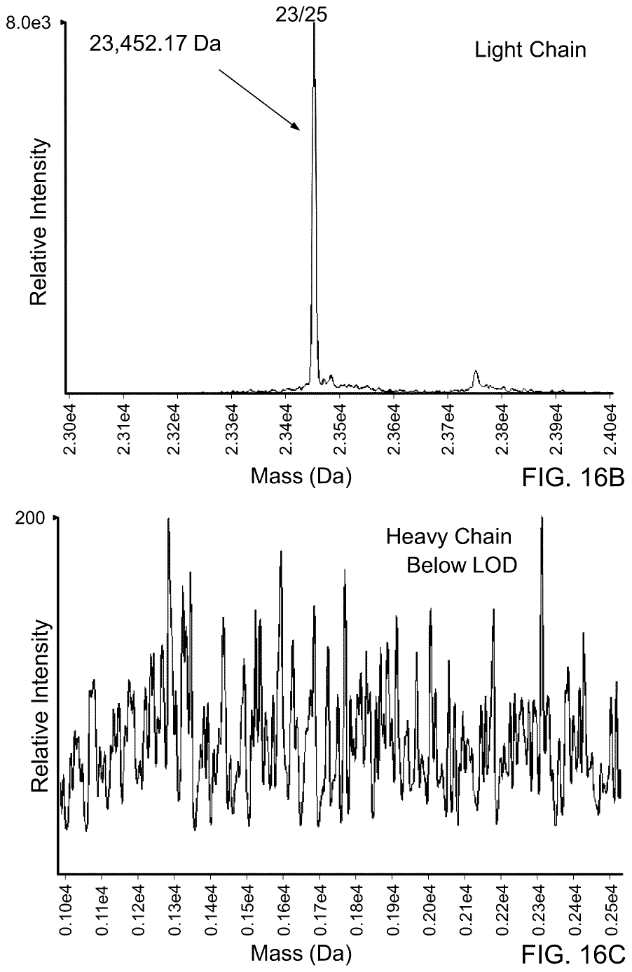

A series of samples from a patient diagnosed with IgG kappa multiple myeloma was examined. The mass spectrum from a serum sample is shown in FIG. 1. The spectrum in FIG. 1A represents a portion of the summed mass spectra across the immunoglobulin LC peak and shows a series of multiply charged ions. The converted molecular mass is shown in FIG. 1B and was calculated to be 23 452.64 Da, representing the proposed molecular mass of the kappa light chain portion of the M-protein. The spectrum in panel C shows another portion of the summed immunoglobulin LC peak and displays a different series of multiply charged ions. The molecular mass for this series shown in FIG. 1D was found to have two components, one at 51 596.07 Da and another at 51 758.27 Da, both of which are consistent with IgG heavy chain. The difference of 162.20 Da between series 2 and 3 may be due to two heavy chain proteoforms differing by the number of hexose units (average MW, 162.14 Da) in the carbohydrate chain. Additional samples taken over a 7 year period were available for testing from this patient. FIG. 16 shows the result using mass spectrometry for a sample taken after the patient had been treated for multiple myeloma and was found to be negative by PEL, IFE, and the quantitative FLC immunoassay. However, multiply charged ions from the light chain are clearly evident in the mass spectrum shown in FIG. 16A. After conversion to molecular mass, a distinct peak at 23 452.17 Da is observed that differs by 0.47 Da compared to the value calculated in the spectrum from the initial diagnosis sample taken over 6 years earlier. Table 1 lists a summary of the results of monitoring the M-protein in serum by PEL, IFE, and microLC-ESI-Q-TOF MS and shows that the light chain is observed throughout the sampling dates, including all of the dates where PEL and IFE were negative. Also, the molecular mass of the light chain remains consistent with an average value of 23 452.54 Da and a standard deviation of 0.86 Da for molecular mass calculations over the 7 year sample period. The heavy chain was observed in the PEL and IFE positive samples, and in those samples, the molecular mass calculations were consistent over the 7 year period. This supports the assumption that M-protein molecular mass is a highly sensitive marker of the plasma cell clone. In addition, a linear regression analysis was done to evaluate the correlation in response between the M-spike value and the peak areas from deconvolution. The correlation for the light chain was r2=0.9455, whereas the two heavy chain proteoforms had r²=0.9205 and 0.9222, respectively.

Additional experiments were performed using matched urine and serum samples taken from a patient with a known monoclonal gammopathy to determine if the molecular mass of the monoclonal light chain would remain constant after being excreted through the kidney into the urine. Two patients (one IgA kappa and one IgA lambda) who had previously been identified as having a monoclonal gammopathy were examined. However, the samples analyzed by microLC-ESI-Q-TOF MS were negative by PEL and IFE for a monoclonal immunoglobulin in both serum and urine. Our findings showed that the molecular mass of the light chain did not change between serum and urine within the expected mass error of the experiment. These results reinforce the principle that molecular mass alone can be used to monitor a monoclonal immunoglobulin regardless of the sample type and that microLC-ESI-Q-TOF MS is a method for identifying a monoclonal immunoglobulin in urine.

TABLE 1

| | | | light chain | | heavy chain | |
|---|---|---|---|---|---|---|
| Sampling Date | M-spike (g/dL) | IFE | peak area | molecular mass (Da) | molecular mass (Da) | |
| Feb. 23, 2005^{b} | 4.35 | pos | 3 010 899 | 23 452.64 | 51 595.07 | 51 758.27 |
| Mar. 29, 2006 | 0.26 | pos | 34 839 | 23 452.10 | | |
| Apr. 24, 2007 | 0 | neg | 9 301 | 23 451.78 | | |
| Oct. 31, 2007 | 0 | neg | 11 496 | 23 452.31 | | |
| Apr. 23, 2008 | 0.54 | pos | 152 021 | 23 452.20 | 51 596.46 | 51 757.84 |
| May 7, 2009 | 0.43 | pos | 323 375 | 23 452.34 | 51 596.66 | 51 758.52 |
| Jul. 27, 2010 | 3.24 | pos | 3 121 072 | 23 452.50 | 51 596.56 | 51 758.91 |
| Aug. 22, 2011^{c} | 0 | neg | 2112 | 23 452.17 | | |
| Mar. 5, 2012 | 0.79 | pos | 600 281 | 23 452.50 | 51 596.44 | 51 758.74 |

^{a}Results are from serum samples obtained from a patient diagnosed with muliple myeloma taken over a 7 year period.
^{b}Sample date Feb. 23, 2005 was used in FIG. 2
^{c}Sample date Aug. 22, 2011 was used in FIG. 3.

### Example 6. Identification of Light Chain Isotype by Top-Down MS

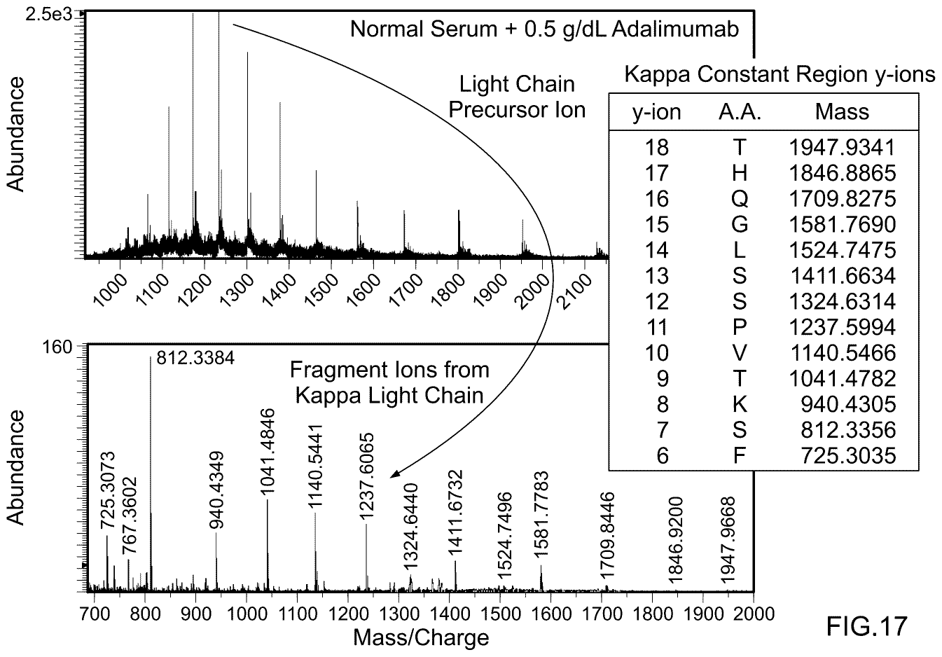

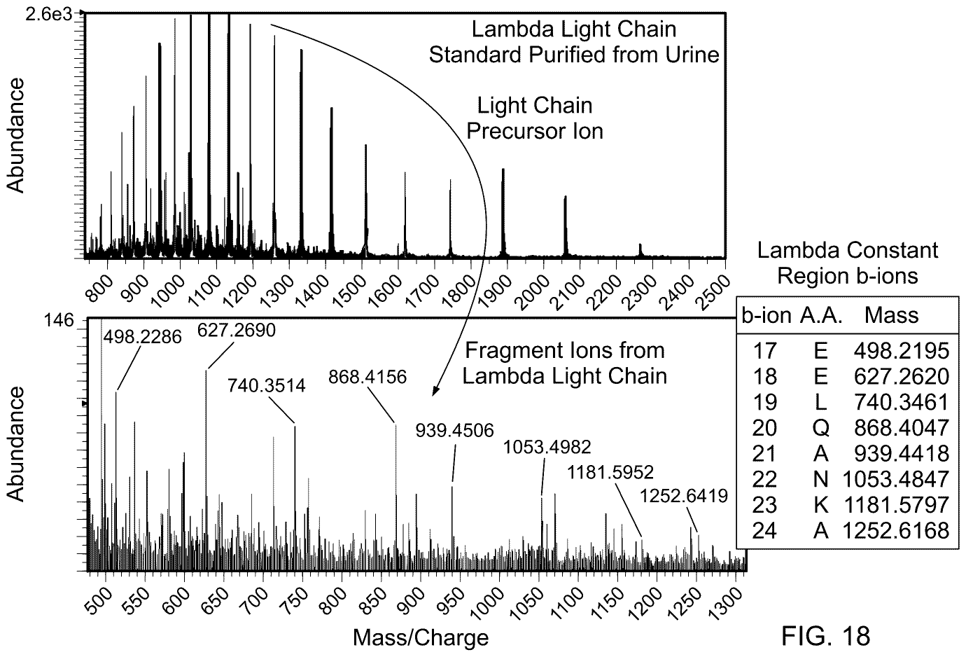

Top-down MS was done on a multiply charged ion from the light chain of adalimumab, which has a kappa isotype. The results from a top-down analysis using adalimumab spiked into normal serum are shown in FIG. 17. FIG. 17 shows the multiply charged ions from the kappa light chain along with an arrow to the fragment ion mass spectrum produced from the CID of the precursor at m/z=1233 shown in FIG. 17. Fragment ions are labeled with their monoisotopic masses, which closely match the calculated monoisotopic masses for y ions from the constant region of the kappa light chain. To determine if this would hold true for other patients with kappa light chains, a set of 20 IgG kappa patients was analyzed by top-down MS on the light chains. The fragment ion mass spectrum for each patient was generated from a different multiply charged precursor ion because of an individual's different variable region amino acid sequence. However, regardless of the patient-specific precursor ion, the same y ions matching the kappa constant region were identified. All 20 of the patients tested showed the same kappa-specific fragment ions. LC-ESI-Q-TOF MS experiments were also performed on a commercially available lambda light chain. The mass spectrum on the top of FIG. 18 shows the multiply charged lambda light chain with the fragment ion spectrum on the bottom of FIG. 18. Initial comparison between the monoisotopic masses for the observed fragment ions and the potential y ions from the 5′ lambda constant region did not produce matches. The topdown MS protein database search engine ProSight was used to search the fragment ions to find a possible match within the constant region. The table on the right of FIG. 18 lists the lambda light chain constant region sequence tag found by ProSight along with the monoisotopic masses for b ions from the sequence. Fragment ions that match the monoisotopic masses in the table are labeled in the spectrum. Although the intensity of the b-ion series for lambda may be less pronounced than those observed for kappa, the fragment ions observed are unique to the lambda light chain isotype. A set of 20 patients positive for an IgG lambda light chain was analyzed by top-down MS fragment, and the b ions matching the N-terminal portion of the lambda constant region were observed in each patient. In addition, IFE positive urine samples were analyzed by top-down MS and found that lambda-positive samples had lambda-specific fragment ions and kappa-positive samples had kappa-specific fragment ions. These findings led us to conclude that top-down MS could be used to isotype kappa and lambda light chains.

The results shown here provide the empirical evidence to substantiate the utility of mass spectrometry as a tool to monitor an M-protein in patients with a monoclonal gammopathy. The molecular mass of the monoclonal immunoglobulin, whether it is the light chain, heavy chain, or the intact molecule, represents a sensitive and specific marker of immunoglobulin-secreting plasma cell clones. The methodology can readily identify a monoclonal immunoglobulin present above the polyclonal background, providing exceptionally detailed information about the status of patient-specific plasma cell clones. In the future, mass spectrometry might play an important role in the quantitation and monitoring of immunoglobulins in human health and disease.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1-30. (canceled)

31. A method of detecting a monoclonal kappa or lambda light chain immunoglobulin expansion in a sample, comprising:
subjecting a sample comprising intact immunoglobulin kappa or lambda light chains to tandem mass spectrometry; and
detecting the presence of the kappa or lambda light chain immunoglobulin expansion by measuring constant region fragment ions produced by collision induced dissociation.

32. The method of claim 31, wherein the sample is a whole blood, serum, plasma or urine sample.

33. The method of claim 31, wherein intact immunoglobulins are isolated from the sample to obtain a mixture comprising different immunoglobulin species prior to subjecting the sample to tandem mass spectrometry.

34. The method of claim 33, wherein the isolating step comprises chemical-based fractionation or affinity purification.

35. The method of claim 33, wherein the intact immunoglobulins are decoupled prior to subjecting the sample to tandem mass spectrometry.

36. The method of claim 35, wherein the decoupling comprises treating the intact immunoglobulins with a reducing agent.

37. The method of claim 31, wherein the tandem mass spectrometry comprises a first mass spectrometry technique employing an electrospray ionization (ESI) source to obtain a mass spectrum comprising a distribution of charged light chain ion peaks.

38. The method of claim 37, wherein the charged light chain ion peaks are charged intact light chain ion peaks.

39. The method of claim 38, wherein the charged intact light chain ion peaks are multiple charged intact light chain ion peaks having a molecular mass of about 21,000 Da to about 26,000 Da.

40. The method of claim 39, wherein the molecular mass is determined by deconvolution of the multiple charge states.

41. The method of claim 31, wherein the monoclonal kappa or lambda light chain immunoglobulin expansion is an endogenous monoclonal kappa or lambda light chain immunoglobulin expansion associated with a monoclonal gammopathy.

42. The method of claim 41, wherein the monoclonal gammopathy is multiple myeloma.

43. A method for determining whether a monoclonal light chain immunoglobulin is a kappa light chain or a lambda light chain, comprising:
subjecting a sample comprising intact immunoglobulin light chains to tandem mass spectrometry; and
comparing m/z's of one or more fragment ions produced by collision induced dissociation to m/z's that are expected to result from the constant region of the kappa light chain or the lambda light chain

44. The method of claim 43, wherein the sample is a whole blood, serum, plasma or urine sample.

45. The method of claim 43, wherein intact immunoglobulins are isolated from the sample to obtain a mixture comprising different immunoglobulin species prior to subjecting the sample to tandem mass spectrometry.

46. The method of claim 45, wherein the isolating step comprises chemical-based fractionation or affinity purification.

47. The method of claim 45, wherein the intact immunoglobulins are decoupled prior to subjecting the sample to tandem mass spectrometry.

48. The method of claim 47, wherein the decoupling comprises treating the intact immunoglobulins with a reducing agent.

49. The method of claim 43, wherein the tandem mass spectrometry comprises a first mass spectrometry technique employing an electrospray ionization (ESI) source to obtain a mass spectrum comprising a distribution of charged light chain ion peaks.

50. The method of claim 49, wherein the charged light chain ion peaks are charged intact light chain ion peaks.

51. The method of claim 50, wherein the charged intact light chain ion peaks are multiple charged intact light chain ion peaks having a molecular mass of about 21,000 Da to about 26,000 Da.

52. The method of claim 51, wherein the molecular mass is determined by deconvolution of the multiple charge states.
